(19) United States
(12) Patent Application Publication
(10) Pub. No.: US 2009/0222000 A1
(43) Pub. Date: Sep. 3, 2009

(54) DEVICE AND METHOD FOR THE TREATMENT OF TUMOURS

(75) Inventor: Andrew Pacey, Hertfordshire (GB)

Correspondence Address: BROOKS KUSHMAN P.C., 1000 TOWN CENTER, TWENTY-SECOND FLOOR, SOUTHFIELD, MI, 48075, US

(73) Assignee: EMCISION LIMITED, London (GB)

(21) Appl. No.: 12/281,878

(22) PCT Filed: Mar. 5, 2007
(86) PCT No.: PCT/GB07/00768

§ 371 Date: Nov. 21, 2008

(30) Foreign Application Priority Data
Mar. 6, 2006 (GB) 0604471.3

(51) Int. Cl. A61B 18/18; A61B 17/32

(52) U.S. Cl. 606/33; 606/186

(57) ABSTRACT

A device includes an applicator and electrode needles forming two rows. Needles may be inserted into tissue and electromagnetic power may be applied thereto, following which a blade may be used to cut the tissue. The blade may be located between or outside and parallel to needle rows. The blade is located on an actuator operable by a handle, which may be in the form of a rod.

The present invention relates to a device and method for the treatment of and in particular the removal of tumours, including tumours within a body of tissue such as the liver which will bleed profusely when cut.

When tumours occur within a body of tissue having a heavy blood supply, such as the liver, surgical removal of the tumour by resection has to be undertaken with the greatest of care if significant and potentially life threatening blood loss is to be avoided. Conventionally, liver surgery involving resection is carried out as an open procedure, with the surgeon being required to tie off or to apply localised heating to seal each of the blood vessels within the cut surface. It will be understood that this is a long and difficult procedure, and in recent years other approaches such as ablation have become more popular. In this context, ablation consists of inserting into the centre of the tumour one or more thin needles, and then heating those needles, for example using applied RF energy, to kill the tumour from the inside. Once the tumour has been entirely killed, it can simply be left in place, thereby obviating the need for resection. A typical prior art device for this purpose is disclosed in U.S. Pat. No. 6,660,002.

Unfortunately, there are a number of problems associated with this approach. It is difficult for a surgeon to tell whether or not all parts of the tumour have been killed. The heating effects of devices such as that disclosed in U.S. Pat. No. 6,660,002 are non-uniform, leading to concern that some cancerous cells may not be heated up sufficiently to kill them. Such areas are most likely to occur adjacent to or within larger blood vessels, since the blood itself will act as a medium for carrying heat away from those areas and thus cooling them. It will be understood that the consequence of leaving in place live cancerous cells which are adjacent to or within a major blood vessel is particularly dangerous, since it is those cells having good blood supplies that are most liable to continue growing, and indeed to continue growing rapidly.

The problems associated with ablation devices such as that disclosed in U.S. Pat. No. 6,660,002 are greatest for larger tumours, of diameter greater than 3 cm. With a large tumour, it may take far too long for the heat to spread from the centre to the outer periphery of the tumour. It is, of course, always open to the surgeon to use the device on individual larger sections of the tumour in turn; however this is typically quite time consuming and runs the risk that smaller areas may inadvertently be missed.

To overcome the risks associated with ablating a tumour and leaving it in situ, it is desirable to reliably isolate a tumour within a body of tissue and preferably remove it, without reverting to time consuming and difficult traditional resection procedures. EP-A-1100585 discloses a device for generating localised heating in a selected region of body tissue, prior to the surgical incision of that tissue. The device comprises an array of needles which are guided into the tissue or organ being treated. Microwave energy is used to raise the temperature of the selected tumour or region of tissue. Once the tumour has been isolated in this manner, if it is to be excised the ablation device must be withdrawn from the tissue and a separate manual cutting device must be inserted to remove the tumour.

According to a first aspect of the present invention there is provided a device for the treatment of tumours or other tissue comprising an applicator, a plurality of needles extending from the applicator, and a blade arranged for movement longitudinally along the needles.

The needles may be configured in a generally square or rectangular array of N×M needles, where N and M are integers. N may, for example, be 1 or 2. M may, for example, be 2 or greater, such as a number between 2 and 8, for example 2 or 3.

According to a second aspect of the present invention there is provided a method of treatment comprising:
(a). Using an applicator to extend into an area of tissue a plurality of needles;
(b). Applying electromagnetic power to the needles to heat and embolise the said area of tissue; and
(c). Urging a blade longitudinally along the needles to cut into the said area of tissue.

A number of preferred features are set out in the dependent claims.

The present invention finds particular although not exclusive application in the removal of tumours within highly vascular tissues such as for example the liver, the breast, the bone, the lung, the kidney, the pancreas, the spleen or the uterus. Optionally, the device and method will be used in conjunction with a suitable imaging system such as for example ultrasound, X-Ray, MRI, or CT.

The needles may be diamond section in shape and, depending on the energy source being supplied, may act either as a microwave array or RF electrodes, whereby, for RF, adjacent needles may be of similar or opposing polarities. The placing of the needles may be verified by imaging and the tissue surrounding or rear the tumour then irradiated with electromagnetic radiation of RF or microwave frequency, causing the collagen surrounding the blood vessels to constrict and the blood to coagulate.

Having created a plane of a vascular tissue, the target tissue (such as at or near a tumour) may be safely cut by the blade. By choosing the correct size of device and number of needles used, a minimum amount of healthy tissue can be removed around the tumour. Once the device has been used to isolate and remove all of the target tissue, the remaining void may be inspected by any suitable optical means.

The device and method of the present invention allow surgeons to safely and efficiently isolate and remove tumours from surrounding tissue, in particular highly vascular tissue, without the need to individually seal each blood vessel surrounding the tumour or use a plurality of operating instruments.

Embodiments of the invention will now be described, by way of example, with reference to the drawings of which:

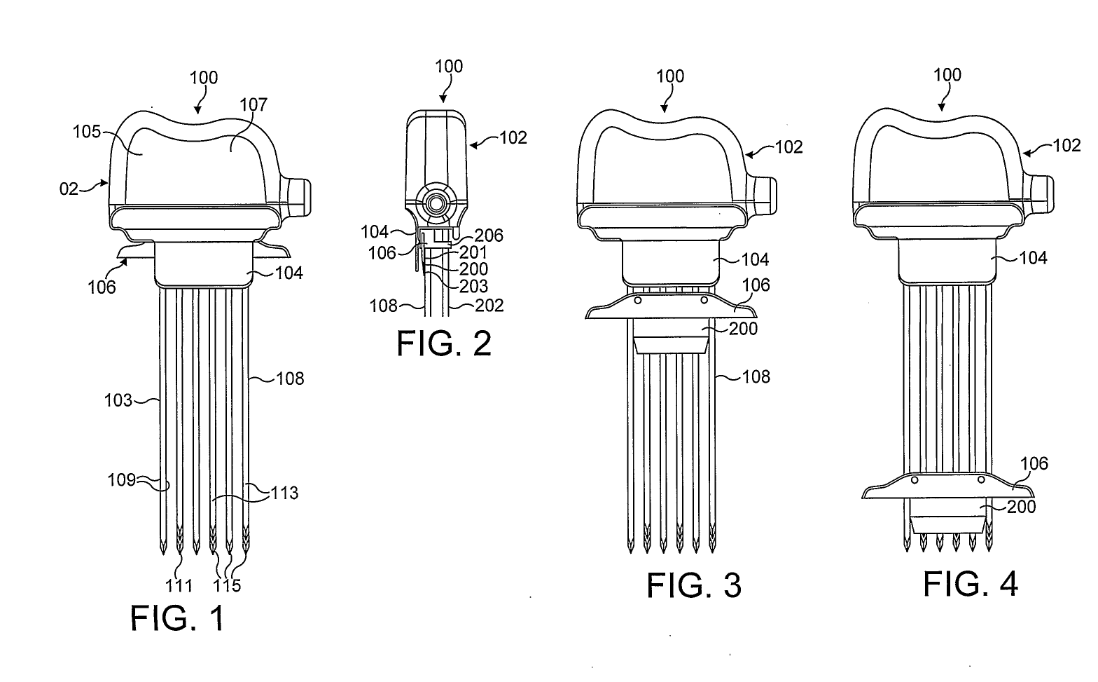

FIG. 1 shows a front view of the device, with the blade covered by the shell;

FIG. 2 shows a side view of the upper part of the device of FIG. 1;

FIG. 3 shows a front view of the device, with the blade lowered below the shell;

FIG. 4 shows a front view of the device with the blade lowered for incision purposes;

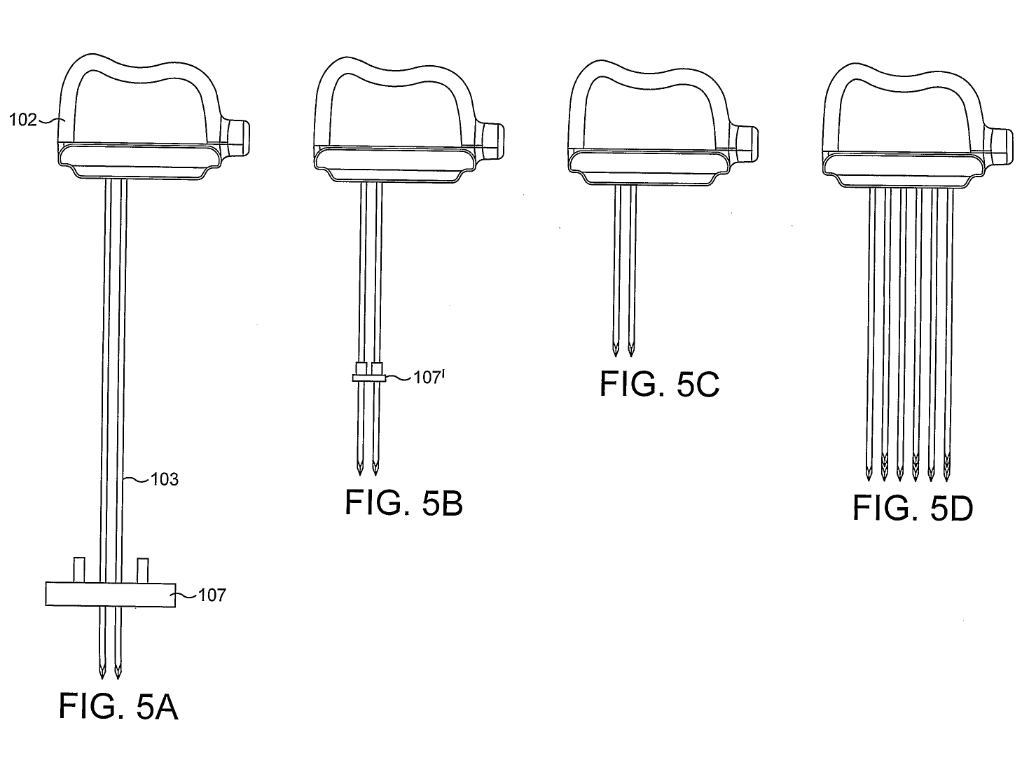

FIG. 5A to 5D shows several front views of the device, in which different lengths of needles are employed in conjunction with the same applicator;

Referring to FIGS. 1 and 2, the device 100 may be seen in more detail. The device comprises a manually-graspable applicator 102 from which a plurality of needles 103 extend. Applicator 102 thus has a preferably including recesses or concave main handle portion 105, portion 105 contoured portions 107 which may be on opposite faces thereof enabling easy grasping of applicator 102. The needles may optionally be divided into a first row 108 and a second row 202 in order to define an area of tissue for heating. Extending from the front face of the applicator 102, outward of the first row 108 of needles, is a blade cover or shell 104 which is arranged to cover a blade 200 when it is not in use. Also partially covered by the shell 104 in this position is a blade holder 106 from which the blade 200 extends and which optionally acts as a connector and guide for the first 108 and second row 202 of needles. The blade 106 acts as an actuator, manual pressure on which forces the blade downwardly.

As can be seen from FIG. 2, the blade holder 106 has a shelf 206 which extends rearwardly and which connects the first 108 and second 202 rows of needles. The needles extend from the applicator 102 through the shelf 206 of the blade holder 106 which acts as a needle assisting to define the separation between the two rows of needles and hence defining the width of the tissue region which is to be heated.

In use, the needles are pushed into an area of tissue to be resected, for example the tissue adjacent a tumour. Typically, although not necessarily, the device will be used to cut into healthy tissue immediately adjacent a tumour or other tissue to be resected. The tumour itself is not cut into.

Once the needles are in place, electromagnetic energy is supplied to two adjacent needles, causing heating and embolisation of the segment of tissue between the two needles. Once that tissue has been heated to a sufficiently high temperature for a sufficient period of time to embolise any blood vessels, the power is switched off and is reapplied between the adjoining pair of adjacent needles. The process is repeated until, segment by segment, the whole of the peripheral tissue surrounding the tumour has been embolised. It will be appreciated that this process may be automated and carried out under computer control, and that the electromagnetic energy may, if desired, be applied via more than two needles at once.

It will be further appreciated that there are numerous ways in which the tissue S adjacent the needles may be embolised. If sufficient power is available, it may be preferable to heat up all of the individual segments between the needles at once. Any appropriate form of electromagnetic energy may be used to heat the tissue, for example RF or microwave energy. If RF is used the individual needles may be monopolar or bipolar. A control system (not shown) may be provided for operating adjacent needles with opposing polarity. If microwave energy is used, the needles may form a microwave array which would contain the microwave energy largely within the selected region of tissue.

Once the blood flow has been occluded within the peripheral area surrounding the tumour, for example after more than one application of the device with its needles in differing locations around at or near the tumour, any internal tumour must then die for lack of a blood supply. In many circumstances, however, it will still be desirable or necessary to remove the tumour, which can be achieved using the present invention.

As can be seen in FIGS. 3 and 4, at any point during a procedure and in particular once the tumour or other selected region has been fully or partially occluded from the surrounding tissue, the surgeon can choose to push the blade 200 downwards towards the area of tissue to be cut. The blade 200 may be controlled manually by applying pressure to the blade holder 106 or by any appropriate automated or manual actuation means (not shown). Once the blade 200 is at the appropriate level with respect to the tumour or other selected tissue, it can then cut the tissue at that point thereby at least partly excising the tumour from the surrounding healthy tissue. When it is not in use, the blade 200 may be repositioned behind the shell 104.

The device may be applied multiple times, as required, to cut around the periphery of a tumour, for example with the blade 200 being operated after application of electromagnetic power before moving the needles to a new location before repeating the procedure of application of power, cutting using the blade 200, then repositioning, and so on.

Generally, the needles of the device will be disposed mutually parallel, for example in first 108 and second 202 rows as described above. They may be formed of steel or any other appropriate material. As can be seen in FIGS. 1, 3 and 4, the needles may have main circular sectioned portions 109 and sharpened diamond section tips 111 to aid their passage through the tissue. In other embodiments, the needles may have a diamond shaped section along their whole length. There may be more, or fewer needles than are shown in the drawings, and the needles need not be arranged in straight parallel rows. As shown in, for example, FIG. 1, the needles 108 may have varying lengths, such as by having alternating lengths along each row with needles being eight short needles 113 or long needles 115, each needle in the array of needles being either longer or shorter than all of its adjacent needles. This arrangement assists in insertion of the needles into tissue by concentrating initial insertion force over a smaller area. As shown in FIG. 5D the larger needles in this embodiment are 5.07 mm larger than the shorter needles.

The blade 200 may also be formed of steel or any other appropriate material, such as a ceramic. As can be seen best in FIG. 2, the blade 200 may comprise an upper portion 201 of uniform width and a lower portion 203, the width of which decreases steadily to define a sharp point at the tip of the blade.

Optionally, the needles may be at least partially retractable into the applicator 102. This enables the device 100 to use needles of a given length for insertion at a variety of depths within the tissue. Alternatively or in addition, as shown in FIGS. 5A to 5C in which blade 200 and shell 104 are not shown for clarity purposes, a variety of needle lengths may be used in conjunction with the applicator 102, depending on the depth of tissue being embolised. In FIG. 5A, four needles 103 extend 200 mm fan applicator 102 and a guide 107 may be provided for maintaining needles spaced and parallel or for providing a guide for needle insertion depth. Cutting edge 205 of blade 200 therefore extends past the below guide 107 when blade 200 is used for cutting. FIG. 5B shows four mid-size 110 mm needles with guide 107. FIG. 5C shows for 65 mm needles 103. FIG. 5D shows twelve needles, half being 110 mm and half slightly shorter.

The needle 103 shown in FIGS. 5A to 5D may be added to or removed from the applicator to provide the desired configuration. It will be understood that the present invention may be used for generating localised heating in a selected region of tissue and/or for the excision of a selected region of tissue from the surrounding tissue. The dual functionality of the device removes the need for using separate ablation and cutting instruments in surgical procedures. This provides the advantage of increased efficiency by reducing the number of instruments which a surgeon must manipulate during a surgical procedure. In addition, using a single device reduces the time taken to perform a particular procedure and minimises the number of times any region of tissue is invaded.

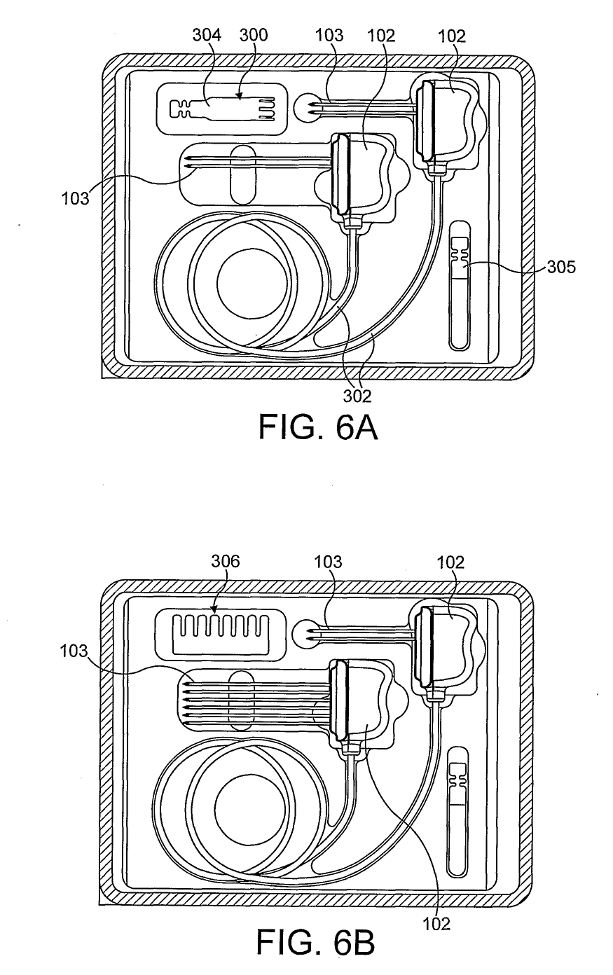

As shown in FIG. 6A, a blister pack 300 may contain two or more applicators 102 connected to arrays of needles 103 having different configurations for ease of use. A wire 302 is fitted to each applicator 102 for the provision of electromagnetic power thereto. The blister pack 300 may also contain a tool 304 and tool 305. Similar blister pack 308 shown in FIG. 6B includes applicators 102, one of which has four needles 103 attached thereto like both of the applicators shown in FIG. 6A, the other having 12 needles 103 in two parallel rows of six needles. Blister pack 308 includes a comb tool 306. The blade 200 and shell 104 are not shown in FIGS. 6A and 6B for the purposes of clarity. Tools 304, 305, 306 may be used to clean or wipe tissue off needles 103, and also serve as combs and ‘push off’ plates used to (a) ensure correct spacing on needle entry to reduce the chance of needle convergence during insertion, and (b) to reduce the risk of tearing tissue on removal of the device as force for withdrawal is spread across the tissues, and (c) they are also used for cleaning needles without removing the coatings.

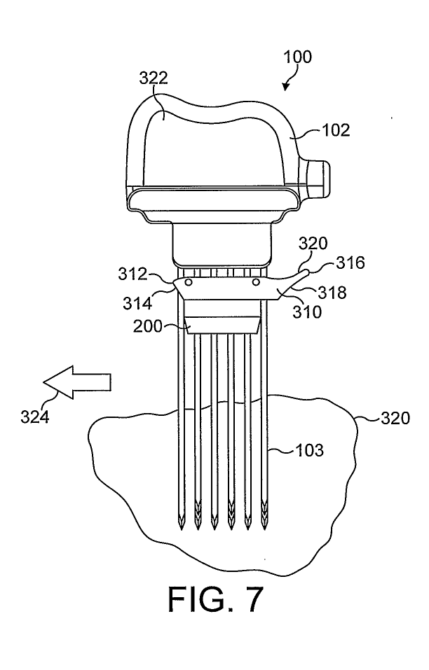

FIG. 7 shows a modification to the device 100 shown in FIGS. 1 to 4 in which the blade holder 106 is replaced with a revised blade holder 310 which may be more conveniently operated by the surgeon than the blade holder 106 of FIGS. 1 to 4. In particular, on one side 312 thereof, the blade holder 310 has a ramp surface 314, and the other side 316 of the blade holder 310 as a corresponding ramp surface 318 formed as part of a manually operable blade actuation handle 320. The ramp surfaces 314, 318 enable the blade 200 to be easily pushed along the needles 103 with a cutting motion into tissue 320 (shown schematically in FIG. 7) after application of electromagnetic powers to the needles 103 and before removal of the needs 103 from the tissue 320. The rearwardly extending handle 320 which extends rearwardly towards the applicator 102 and the applicators manually graspable handle 322 enable the surgeon to withdraw the blade 200 again. Although the surgeons method of operation of the device 100 may vary according to the required procedure, for example when carrying out a liver resection, the surgeon may insert the needles 103 into the tissue 320, apply appropriate electromagnetic power, then cut using the blade 200, then repeat the operation such as by moving to the left as shown by arrow 324 in FIG. 7, using the needles to ablate and coagulate the tissue and the knife 200 thereafter to cut the tissue without excessive bleeding upon each needle application and cutting cycle, and the developing procedure may therefore result in a growing cut through the tissue such as a planer or generally curved cut as may be required by the surgeon.

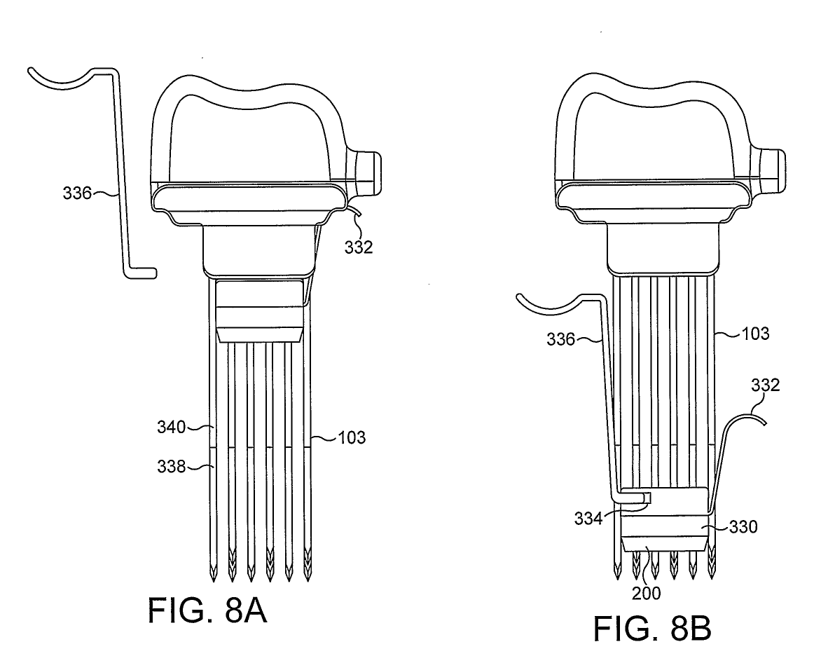

FIGS. 8 and 8B show a further modification in which the blade holder 330 is similar to the blade holder 310 shown in FIG. 7. However, the blade holder 330 is provided with a rearwardly extending hook 332 which is manually operable by the surgeon for pushing the blade 200 with a cutting motion into tissue, and for pulling the blade 200 back from the extended position shown in FIG. 8B to the retracted position shown in FIG. 8A. Additionally, blade holder 330 is provided with a slot 334 to which an auxiliary operating handle 336 may be optionally removably coupled for sliding the blade 200 along the needles 103, such as when needles 103 are deeply inserted into tissue 320.

As shown in FIGS. 8A and 8B, the tip portions B38 of needles 103 may conduct into tissue and upper portions 340 needles may be insulated.

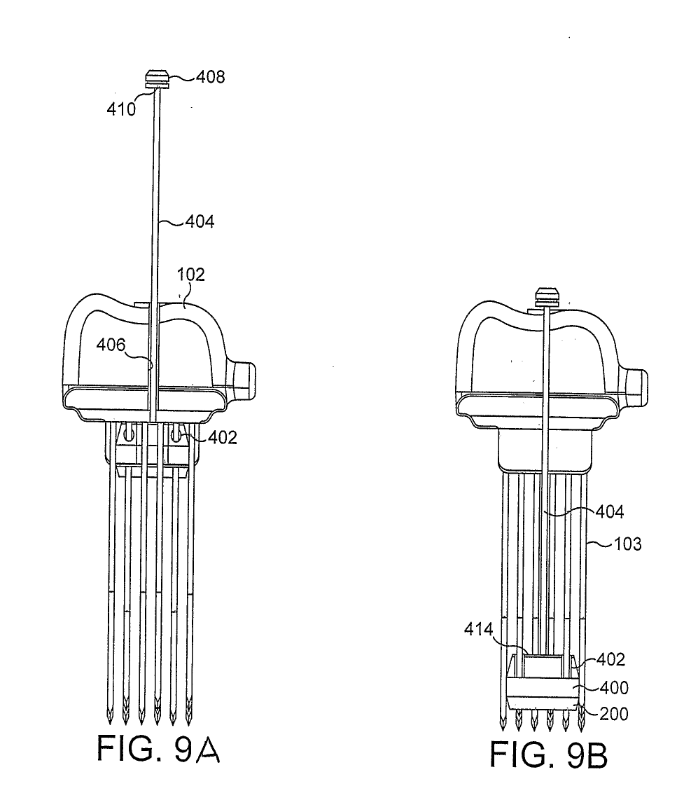

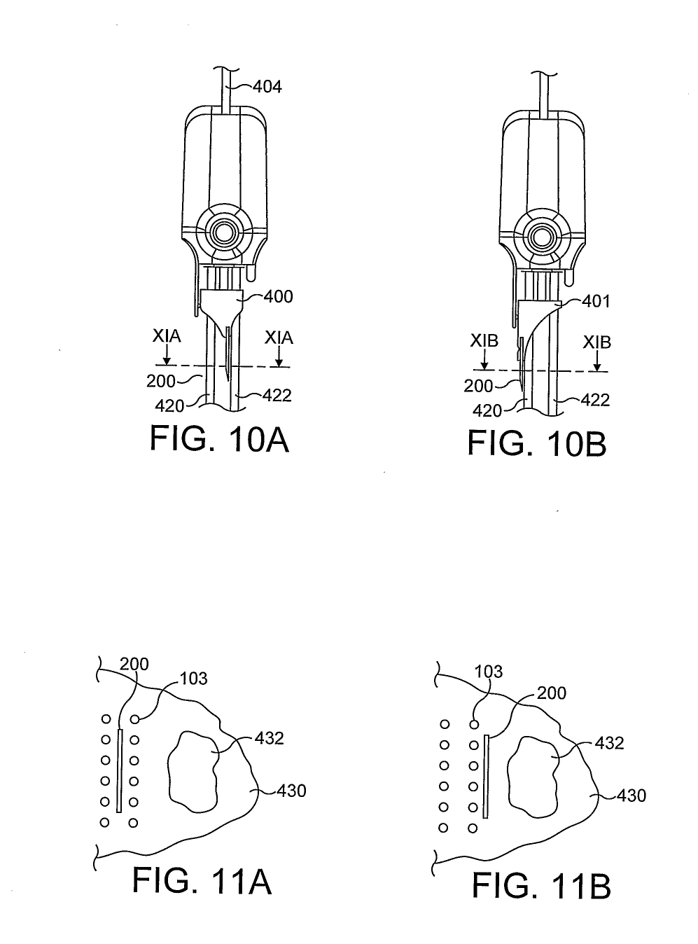

FIGS. 9A, 9B and 10A show a further modification in which blade holder 400 has bores 402 (8 bores) enabling sliding of the blade 200 along the needles 103. Blade holder 400 has a rearwardly extending rod 404 which extends through a bore 406 in applicator 102 and is operable by a manually operable knob 408 on the end thereof 410 opposite the blade holder 400. This configuration allows the blade to be conveniently slid along the needles 103 without the surgeon having to perform manual operation of the blade 200 with a hand forward of the applicator 102 towards the tissue into which the needs are inserted, thereby providing good visibility from the surgeon. Also rod 404 is centrally located on rear surface 414 of blade holder 400 thereby avoiding unnecessary application of bending moments to needles 103 and minimum friction during the sliding movement of the blade holder 400 along the needles 103. As shown in FIG. 10A, the twelve needles 103 are in two parallel rows 420, 422 and knife 200 is parallel with and longitudinally slidable along the rows 420, 422, but if located between the rows 420, 422. FIG. 11a which is a section on plane XIA in FIG. 10A, shows how needles 103 may be inserted into healthy tissue 430 and the knife 200 used to cut tumour 432 off tissue 430, such as during a liver resection operation, such as by repeatedly inserting needs 103 into tissue 430 to resect along a plane or curve as described above. In a modification of the embodiment of FIGS. 9A, 9B and 10A which is shown in FIGS. 10B and 11B, blade 200 may be located using blade holder 401 parallel to an outside needle rows 420, 422, with one needle row 420 between blade 200 and other needle row 422.

Various modifications may be made to the embodiments described herein without departing from the scope of the invention as defined by the claims hereto as interpreted under Patent Law.

1.-28. (canceled)

29. A device for the treatment of tumours or other tissue comprising an applicator, a plurality of needles extending from the applicator, and a blade arranged for movement longitudinally along the needles.

30. A device as claimed in claim 29 in which the applicator has a manually graspable configuration.

31. A device as claimed in claim 29 including a manually operable actuator for moving the blade longitudinally along the needles.

32. A device as claimed in claim 29 in which the needles are arranged to define an area adjacent a tumour or other region of tissue to be treated, the needles being arranged to cause heat to embolise the said area of tissue.

33. A device as claimed in claim 29 in which the needles are arranged to act as electrodes to which electromagnetic power is applied.

34. A device as claimed in claim 33 in which adjacent needles within the device are arranged as electrodes with opposing polarities.

35. A device as claimed in claim 29 in which the needles are arranged to act as a microwave array to which microwave power is supplied.

36. A device as claimed in claim 29 in which the needles are retractable into the applicator.

37. A device as claimed in claim 31 in which the actuator comprises a blade holder.

38. A device as claimed in claim 37 in which the applicator has a blade cover and the blade holder is slidable along the needles to a position in which the blade is covered by the blade cover.

39. A device as claimed in claim 31 in which the manually-operable actuator includes a handle extending rearwardly from a main body of the actuator.

40. A device as claimed in claim 39 in which the handle is selectively connectable to and removable from the main body.

41. A device as claimed in claim 39 in which the main body includes a slot into which a projection of the handle is selectively insertable.

42. A device as claimed in claim 39 in which the handle comprises a rod centrally mounted to the main body of the actuator, the rod extending parallel to the needles and away from tip ends of the needles.

43. A device as claimed in claim 31 in which the actuator includes at least one through-bore and in which the blade is slidable along the needles with a said needle passing through the through-bore.

44. A device as claimed in claim 29 claim which includes two rows of said needles, the blade being located between said needles.

45. A device as claimed in claim 44 in which the blade is closer to one said row than the other.

46. A device as claimed in claim 29 which includes two rows of said needles, one said row being located between the blade and the other side row.

47. A method of treatment comprising:
using an applicator to insert into an area of tissue a plurality of to needles;
applying electromagnetic power to the needles to heat and embolise the said area of tissue; and
urging a blade longitudinally along the needles to cut into the said area of tissue.

48. A method as claimed in claim 47 in which the said tissue comprises healthy tissue adjacent a tumour to be resected.

49. A method as claimed in claim 47 in which the electromagnetic power is RF power.

50. A method as claimed in claim 47 in which the electromagnetic power is microwave power.

51. A method as claimed in claim 47 which includes removing needles from said tissues after cutting said tissue, then reinserting said needles into said tissues at a different location and applying EM power again and cutting using said blade again.

52. A device/method as claimed in claim 29 in which the needles are configured in an array of N×M needles, where N and M are integers, the array having a configuration selected from the group of (a) generally square and (b) generally rectangular.

53. A device/method as claimed in claim 52 in which N is selected from the group of (a) 1 and (b) 2.

54. A device/method as claimed in claim 52 in which M is selected from the group of (a) 2 and (b) greater than 2.
